(19) United States
(12) Patent Application Publication
(10) Pub. No.: US 2004/0259250 A1
(43) Pub. Date: Dec. 23, 2004

(54) Methods and compositions for modifying a receptor tyrosine kinase protein tyrosine kinase signal to an apoptotic signal in a cell

(76) Inventors: Howard Perry, Victoria (CA); Anthony Pawson, Toronto (CA)

Correspondence Address: ROPES & GRAY LLP, ONE INTERNATIONAL PLACE, BOSTON, MA, 02110-2624, US

(21) Appl. No.: 10/469,509

(22) PCT Filed: Mar. 1, 2002
(86) PCT No.: PCT/CA02/00282

Related U.S. Application Data
(60) Provisional application No. 60/272,497, filed on Mar. 1, 2001.

(51) Int. Cl. C12N 15/85

(52) U.S. Cl. 435/455

(57) ABSTRACT

Methods and compositions for modifying the RTK/PTK signal in a cell to an apoptotic signal in a cell. Methods and compositions for activating an apoptotic signal in a cell are described involving linking a RTK/PTK signaling pathway that transduces a RTK/PTK signal in the cell to an apoptotic signaling pathway that transduces an apoptotic signal in the call thereby increasing the apoptotic signal in the cell. A RTK/PTK signaling pathway in a cell may be linked to an apoptotic signaling pathway in the cell by creating a new signaling pathway in the cell comprising signaling molecules of an RTK/PTK signaling pathway that transduce a RTK/PTK signal, and signaling molecules of the apoptotic signaling pathway that promote apoptosis. A RTK/PTK signaling pathway in a cell can be linked to an apoptotic signaling pathway in the cell using a domain of a signaling molecule that regulates the RTK/PTK signal and a domain of a signaling molecule that promotes apoptosis. Applications for the methods and compositions are described.

### FIELD OF THE INVENTION

The invention relates to methods and compositions for modifying a receptor tyrosine kinase (RTK)/protein tyrosine kinase (PTK) signal in a cell to an apoptotic signal in the cell.

### BACKGROUND OF THE INVENTION

Cells grow and respond to their environment through the use of biochemical pathways that relay messages received at the cell surface to the interior of the cell. Each pathway is comprised of many individual protein-protein interactions, which by interacting and modifying one another, translate signals received at the cell surface into a biological response.

Proteins interact with one another in a highly specific manner, which is achieved through the use of discreet structural domains, or modules. The modules are used in different combinations to specify the output of a particular pathway. In cancer, the pathways that control cell growth and differentiation are altered through mutation such that they drive the cell to grow uncontrollably.

To control against abnormal growth or replication of cellular damage, cells also contain pathways that allow them to self-destruct. This process is called programmed cell death, or apoptosis. Although the pathways controlling cell growth and pathways controlling apoptosis regulate diametrically opposite outcomes, these pathways are both modular in composition.

A key class of proteins involved in cell growth are receptor tyrosine kinases (RTKs) and their intracellular counterparts protein tyrosine kinases (PTKs). These molecules recognize external and internal stimuli and then initiate the signal transduction pathways which control cell growth. In cancer cells, RTKs and PTKs are often either over expressed or mutated, and thereby drive the tumor cell to replicate uncontrollably. Although different cancer types exhibit different profiles of activated RTKs and PTKS, deregulated expression of some form of RTKs or PTKs is common among most cancer, and distinguishes cancer cells from normal tissue. It would be desirable if this trait could be used against cancer cells, by altering the signaling pathway activated by RTK's and PTK's to promote cell death or apoptosis.

### SUMMARY OF THE INVENTION

Broadly stated the present invention relates to a method for modifying a RTK/PTK signal in a cell to an apoptotic signal in the cell comprising linking a RTK/PTK signaling pathway that transduces the RTK/PTK signal in the cell to an apoptotic signaling pathway that transduces the apoptotic signal in the cell, thereby modifying the RTK/PTK signal in the cell. In an embodiment of the invention the RTK/PTK signaling pathway is a mitogenic signaling pathway, preferably an oncogenic signaling pathway, and the RTK/PTK signal is a mitogenic signal or an oncogenic signal.

The invention also provides a method for activating or inducing an apoptotic signal in a cell comprising linking a RTK/PTK signaling pathway that transduces a RTK/PTK signal in the cell to an apoptotic signaling pathway that transduces an apoptotic signal in the cell thereby activating or inducing the apoptotic signal in the cell.

A RTK/PTK signaling pathway in a cell may be linked to an apoptotic signaling pathway in the cell by creating a new signaling pathway in the cell comprising signaling molecules of an RTK/PTK signaling pathway that transduce a RTK/PTK signal, and signaling molecules of the apoptotic signaling pathway that promote apoptosis. Similarly, an oncogenic signaling pathway in a cell may be linked to an apoptotic signaling pathway in the cell by creating a new signaling pathway in the cell comprising signaling molecules of the oncogenic signaling pathway that transduce an oncogenic signal, and signaling molecules of the apoptotic signaling pathway that promote apoptosis.

A RTK/PTK signaling pathway in a cell can be linked to an apoptotic signaling pathway in the cell using a domain of a signaling molecule that regulates the RTK/PTK signal, and a domain of a signaling molecule that promotes apoptosis.

The invention also provides a method for modifying a RTK/PTK signal in a cell to an apoptotic signal in the cell comprising administering to the cell a domain of a signaling molecule that regulates an RTK/PTK signal and a domain of a signaling molecule that promotes apoptosis in the cell, in amounts effective to change or modify a RTK/PTK signal to an apoptotic signal in the cell.

A RTK/PTK signaling pathway in a cell can be linked to an apoptotic signaling pathway in the cell by linking or coupling a domain of a signaling molecule that regulates the RTK/PTK signal and a domain of a signaling molecule that promotes apoptosis in the cell. In an aspect of the invention, the domains are coupled in situ. In an embodiment, an SH2 domain or PTB domain is coupled or linked to a death effector domain. In a preferred embodiment, an SH2 domain of Grb is coupled or linked to a death effector domain of FADD.

In an embodiment of the invention a method is provided for modifying a RTK/PTK signal in a cell to an apoptotic signal in the cell comprising administering to the cell a chimeric protein comprising a domain of a signaling molecule that regulates an RTK/PTK signal and a ligand-binding domain, and a chimeric protein comprising a domain of a signaling molecule that promotes apoptosis in the cell and a ligand-binding domain, in amounts effective to change or modify the RTK/PTK signal to an apoptotic signal in the cell.

In an aspect, the invention provides a method for activating or inducing apoptotis in a cell comprising introducing into the cell a first domain of a signaling molecule that regulates an RTK/PTK signal and a second domain of a signaling molecule that promotes apoptosis in the cell in an effective manner to activate or induce apoptotis in the cell. In an embodiment, the first and second domain are associated i.e. there is a stable interaction between the domains. The association between the domains may be non-covalent or it may be covalent, preferably covalent. In a preferred embodiment, an SH2 domain or PTB domain coupled or linked to a death effector domain are introduced into the cell.

One aspect of the invention is directed to a method for enhancing apoptosis in a cell which expresses a receptor of the tumor necrosis factor (TNF) receptor family comprising administering to the cell an amount of a domain of a signaling molecule that regulates a RTK/PTK signal in a cell and a domain of a signaling molecule that promotes apoptosis in the cell. The invention also provides a method of modulating (e.g. enhancing) TNF-family ligand mediated signaling in a cell comprising administering to the cell an amount of a domain of a signaling molecule that regulates a RTK/PTK signal in a cell and a domain of a signaling molecule that promotes apoptosis in the cell.

The invention also contemplates an isolated molecule comprising a domain of a signaling molecule that regulates a RTK/PTI signal in a cell and a domain of a signaling molecule that promotes apoptosis in the cell. The invention also contemplates a chimeric protein comprising a domain of a signaling molecule that regulates a RTK/PTK signal in a cell and a ligand-binding domain for binding to an oligomerizing ligand. Still further a chimeric protein is contemplated that comprises a domain of a signaling molecule that promotes apoptosis in the cell and a ligand-binding domain for binding to an oligomerizing ligand.

The invention also contemplates a complex comprising a molecule, or chimeric protein comprising a domain of a signaling molecule that promotes apoptosis, in association with a caspase, in particular caspase 8 or caspase 9.

Still further the invention provides an antibody specific for an isolated molecule, chimeric protein, or complex of the invention.

In another aspect, an isolated nucleic acid molecule is provided comprising a sequence encoding a domain of a signaling molecule that regulates a RTK/PTK signal in a cell and a sequence encoding a domain of a signaling molecule that promotes apoptosis in the cell. The invention also contemplates isolated nucleic acid molecules encoding a domain of a signaling molecule that regulates a RTK/PTK signal in a cell and a ligand-binding domain for binding to an oligomerizing ligand. The invention also contemplates isolated nucleic acid molecules encoding a domain of a signaling molecule that promotes apoptosis in the cell and a ligand-binding domain for binding to an oligomerizing ligand.

A nucleic acid molecule of the invention may also comprise a sequence encoding a caspase, in particular caspase 8 or caspase 9.

Nucleic acid molecules of the invention may be inserted into an appropriate vector, and the vector may contain the necessary elements for the transcription and translation of an inserted coding sequence. Accordingly, vectors may be constructed which comprise a nucleic acid molecule of the invention, and where appropriate one or more transcription and translation elements linked to the nucleic acid molecule. A vector of the invention can be used to prepare transformed host cells expressing a molecule or chimeric protein of the invention. Therefore, the invention further provides host cells containing a vector of the invention.

A molecule, chimeric protein, or complex of the invention can be produced by recombinant procedures. In one aspect the invention provides a method for preparing a molecule, chimeric protein, or complex of the invention utilizing an isolated nucleic acid molecule of the invention. In an embodiment a method for preparing a molecule or chimeric protein of the invention is provided comprising: (a) transferring a vector of the invention into a host cell; (b) selecting transformed host cells from untransformed host cells; (c) culturing a selected transformed host cell under conditions which allow expression of the molecule and (d) isolating the molecule or chimeric protein. The invention further broadly contemplates a recombinant molecule obtained using a method of the invention.

The present invention also provides pharmaceutical compositions, which contain pharmaceutically effective amounts of an apoptotic domain, RTK/PTK signal domain, molecule, complex, or chimeric protein of the invention, or a nucleic acid encoding an apoptotic domain, RTK/PTK signal domain, molecule, chimeric protein, or complex of the invention, and a suitable pharmaceutical carrier or delivery system.

In yet another aspect the invention provides a method of treating or preventing disease conditions where the affected cells have a defective RTK or PTK (e.g. mutated RTK or PTK or over expressed RTK or PTK) or a defective ligand for the RTK or PTK (e.g. mutated or over expressed ligand) comprising linking a RTK/PTK signaling pathway in the cell to an apoptotic signaling pathway in the cell to activate the apoptotic signal in the cell.

In an aspect of the invention an effective amount of a domain of a signaling molecule that regulates a RTK/PTK signal in a cell and a domain of a signaling molecule that regulates an apoptotic signal to promote apoptosis in the cell are administered. In an embodiment, a molecule, chimeric protein, complex, nucleic acid molecule, or composition of the invention are administered.

The invention also contemplates use of an apoptotic domain, RTK/PTK signal domain, molecule, complex, or chimeric protein of the invention, a nucleic acid molecule encoding an apoptotic domain, RTK/PTK signal domain, molecule, chimeric protein, or complex of the invention, or a composition of the invention for the prevention and treatment of disease conditions where the affected cells have a defective RTK or PTK (e.g. mutated RTK or PTK or over expressed RTK or PTK) or a defective ligand for the RTK or PTK (e.g. mutated or over expressed ligand).

The invention further contemplates the use of an apoptotic domain, RTK/PTK signal domain, molecule, complex, or chimeric protein of the invention, a nucleic acid molecule encoding an apoptotic domain, RTK/PTK signal domain, molecule, chimeric protein, or complex of the invention, or a composition of the invention in the preparation of a medicament for treating or preventing disease conditions where the affected cells have a defective RTK or PTK (e.g. mutated RTK or PTK or over expressed RTK or PTK) or a defective ligand for the RTK or PTK (e.g. mutated or over expressed ligand).

These and other aspects, features, and advantages of the present invention should be apparent to those skilled in the art from the following drawings and detailed description.

### BRIEF DESCRIPTION OF THE DRAWINGS

The invention will now be described in relation to the drawings in which:

FIG. 1A shows the nucleic acid and amino acid sequences of DEDSH2 adaptor molecule comprising the death effector domain of FADD and the SH2 domain of Grb2. (SEQ ID NOs. 1 and 2)

FIG. 1B shows the nucleic acid and amino acid sequences of DEDPTB adaptor molecule comprising the death effector domain of FADD and the PTB domain of Shc A. (SEQ ID NOs. 3 and 4)

FIG. 2. A) NeuNt transformed Rat2 cells were incubated overnight with DEDSH2, DEDR86K, or SH2 bearing adenoviruses. Adaptors were immunoprecipitated with anti-myc antibody and blotted for either tetra-his antibody, or ERB-B2 (Neu) antibody. B) NeuNt transformed Rat2 cells were incubated overnight with DEDSH2, DEDR86K, or SH2 bearing adenoviruses, and immunoprecipitated with anti-myc. Caspase 8 co-immunoprecipitates with DEDSH2 and DEDR86K, but not with SH2 domain alone. C) Expression of DEDSH2, DEDR86K, but not SH2, in NeuNt transformed Rat2 cells produces DNA laddering characteristic of apoptosis.

FIG. 3. Apoptosis in Ntr2 cells is dependent upon caspase 8 activity. A) Ntr2 cells were incubated overnight with DEDSH2, DEDR86K, or SH2 bearing adenoviruses. Top panel shows phase contrast image of apoptosis resulting from expression of the constructs. Bottom panel shows the corresponding GFP fluorescence. B) Ntr2 were incubated with DEDSH2, DEDR86K, or SH2 bearing adenoviruses, and survival (24 hours) in the presence or absence of caspase 8 inhibitor was measured by crystal violet staining. C) Caspase 8 activity in lysates from GFP, SH2, DEDR86K, or DEDSH2 expressing Ntr2 cells.

FIG. 4. Foci derived from transfection of Rat 2 cells with NeuNt are sensitive to apoptosis induced by DEDSH2 expression. A) Rat 2 cells were transfected with NeuNt and incubated with adenoviruses bearing DEDSH2, SH2, or DEDR86K. Individual foci were examined over a 48 hour period by phase contrast microscopy (a,a′,a″, e,e′e″). B) Surrounding monolayer (48 hours) of non transfected Rat 2 cells. C) Survival of parental Rat 2 versus NeuNt transformed Rat 2 cells (Ntr2) was compared between DEDSH2, DEDR86K, and SH2 expressing cells. D) Rat 2 cells were transfected with NeuNt and incubated with TAT-DEDSH2 or TAT-DEDR86K by phase contrast microscopy. Immunofluorescence staining (anti-HA) of Rat2 cells treated with TAT-DEDSH2 or TAT-DEDR86K (bottom panel).

FIG. 5. Clonagenic assay. Ntr2 cells were incubated with adenoviruses bearing GFP, SH2, PTB, DEDR86K, DEDSH2, or DEDPTB for 1 hour and cells were harvested and plated in media containing 0.25% agarose. A) micrographs of colonies that develop after treatment with various adapters. B) after 3 weeks, colonies were stained with MTT and counted.

FIG. 6. A) Phase contrast micrographs of Npc cell line, TWO3, before and after EGF stimulation. EGF stimulation of cells expressing DEDSH2 leads to appearance of apoptotic bodies. B) EGF stimulation produces a decrease in survival of DEDSH2 expressing cells. C) Western analysis of Npc cell lysates showing EGF stimulation also leads to Parp cleavage in the presence of DEDSH2 (top panel). Blots were probed with tetra-his antibody to confirm expression of DEDR86K, and DEDSH2 in these cells (middle panel). Reprobing of the membrane with anti-Grb2 confirmed equal loading of samples. D) Stimulation of TWO3 cells by EGF produces a 6 fold increase in caspase 8 activity.

### DETAILED DESCRIPTION OF THE INVENTION

In accordance with the present invention there may be employed conventional molecular biology, microbiology, and recombinant DNA techniques within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

Glossary

“Apoptosis” refers to a physiological process used by multicellular organisms to dispose of unwanted cells in an orderly manner. A central feature of the process is the permanent containment of cellular material in membranous structures. The resulting apoptotic particles can be phagocytosed without leaking potentially dangerous intracellular enzymes. The process is mediated through apoptotic signaling pathways.

“Apoptotic signaling pathways” refers to pathways that mediate apoptosis that involve multiple extracellular and intracellular signals, integration and amplification of these signals by second messengers, and the activation of the death effector proteases, i.e. caspases. Activation of caspases initiates a proteolytic degradation that results in apoptotic morphology. One well-established apoptotic signaling pathway involves signaling by cell surface death receptors such as TNF receptors, or Fas which through adaptor molecules recruit and activate caspases. Another pathway is initiated by the withdrawal of growth factors and is regulated by the Bcl-2 family of proteins. In this pathway the caspase cascade is triggered by the release of cytochrome c from the mitochondria and activation of Apaf-1. Apoptosis may also be activated through the Jun kinase cascade. (See Dragovich T., et al Oncogene 17:3207-3213, 1998; Gabriel, N. et al, Oncogene 17:3237-3245, 1998; and Baker, S. J. and E. Premkumar Reddy Oncogene 17:3261-3270, 1998 for reviews of apoptotic signaling pathways).

“Apoptotic signal” refers to the stimuli that activate an apoptotic signaling pathway resulting in apoptosis.

“Caspases” refers to a family of cysteine proteases with homology to CED-3 which are critical in generation of apoptotic morphology (Nunex, G. et al, Oncogene 17:3237-3245, 1998). The caspases are synthesized in the cell as inactive precursors composed of four distinct domains: an amino-terminal domain of variable size (termed N-terminal polypeptide or prodomain), a large subunit, a small subunit, and a linker region between the large and small domains flanked by Asp residues (Nicholson and Thornberry, Trends Biochem. Sci. 22:299-306, 1997). Activation of a caspase is induced by removal of the prodomain and linker regions by proteolytic cleavage, and assembly of the large and small subunits into an active enzyme complex. Caspases have been divided into upstream (initiator) and downstream (effector) caspases based on their site of action in the proteolytic caspase cascade. The initiator and effector caspases have different prodomains. Initiator caspases have a long prodomain containing a death effector domain (DED), or caspase recruitment domain (CARD).

Caspases −8, −9, and −10, (possibly −2 and −5) can initiate the caspase activation cascade and are therefore called initiators. Based on the prototypes, caspases −8 and −9, initiators can be activated either by dimerization alone (caspase-9) or by dimerization with concomitant autoproteolysis (caspase-8). The effector caspases −3, −6, and −7 propagate the cascade and are activated by proteolytic cleavage by other caspases. Sequences for the caspases can be found in GenBank, for example, Accession Nos. AAD24962 and AAH02452 show the human sequences for caspase 8 and caspase 9, respectively.

“Death Receptors” refers to the family of receptors involved in apoptotic signaling pathways including the tumor necrosis factor (TNF) receptor family (Baker, S J & E. Premkumar Reddy, Oncogene 1998 Dec. 24;17(25):3261-70). Death Receptors include Fas, TNF-R1 (p55), DR3 (also known as APO3, WSL-1, TRAMP, LARD), DR4, and DR5 (also known as APO2, TRAIL-R2, TRICK 2 or KILLER) that contain a conserved intracellular sequence known as the “death domain”. The receptors associate through the death domain with a number of intracellular signal transduction molecules that themselves contain a death domain. By way of example, the intracellular domain of the activated Fas receptor interacts with an adaptor molecule called Fas-associated death domain-containing protein (FADD, also known as MORT-1). Subsequent apoptotic signal transduction depends on the interaction of the “death effector domain” of PADD with additional downstream molecules. Fas binding to DAXX through the death domain of Fas activates the Jun kinase cascade that may activate apoptosis. In a similar manner, TNF-R1 induces apoptosis through interaction with the adaptor molecule TNFR-associated death domain protein (TRADD). TNF-R1 can also engage an adaptor called RAIDD (also known as CARDD). RAIDD interacts with the death domain of RIP and with a CARD motif of caspase-2 thereby inducing apoptosis.

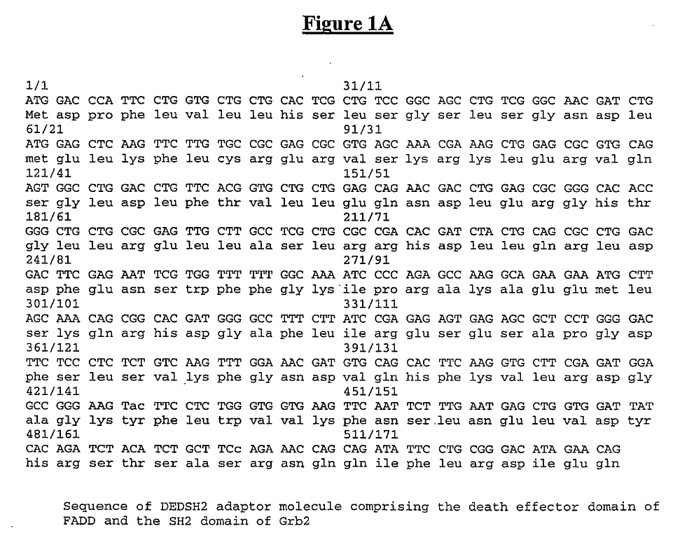

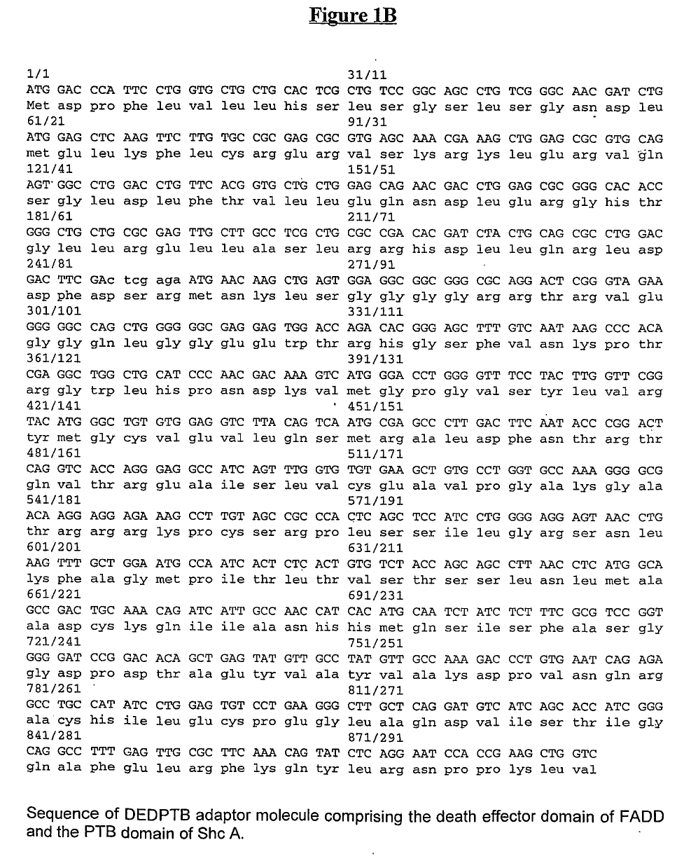

A “domain of a signaling molecule that promotes apoptosis” refers to a modular structural element that transduces an apoptotic signal in an apoptotic signaling pathway resulting in apoptosis. Such a domain is also referred to herein as an “apoptotic domain”. The domain may be from any species, particularly a mammalian species, including bovine, ovine, porcine, murine, equine, preferably the human species, and from any source, whether natural, synthetic, semi-synthetic, or recombinant. Examples of apoptotic domains include the death domain of death receptors, in particular Fas and TNF-R1 and homologues thereof; death effector domains (DED) and caspase recruitment domains (CARD) of caspases and homologues thereof; death domains of adaptor proteins such as FADD, TRADD, RIP, and DAX and homologues thereof; and death effector domains of adaptor proteins (e.g. FADD) including death effector domains that bind to a DED or CARD of a caspase and homologues thereof (see review by K. Hoffman, Cell. Mol. Life Sci. 55: 1113-1128, 1999 and references therein re sequences and structure of death domains, death effector domains, and caspase recruitment domains; particular reference is made to FIGS. 1, 2 and 3 of Hoffman). In some aspects of the invention the term includes proteins or agents containing the domain (e.g. proteins such as death receptors, caspases, adaptor proteins).

“TNF-family ligand mediated signaling” refers to activation or transduction of an apoptotic signal in a cell activated by a TNF-family ligand.

A “TNF-family ligand” refers to a ligand that interacts with a receptor of the tumor necrosis factor (TNF) receptor family thereby inducing an apoptotic signal. TNF-family ligands include TNF, TNF-α, LT-α, Fas ligand (CD95L) OX40L, CD401, CD27L, CD30L, 4-11BBL, LTβ, Apo3L/TWEAK, APO2L/TRAIL, and RANKL (Baker and Premkumar Reddy, Oncogene 17:3261-3270, 1998).

“RTK/PTK signaling pathways” refers to pathways mediated by RTKs or PTKs that involve multiple extracellular and intracellular signals, integration and amplification of these signals by second messengers, and the activation of cellular processes including cell proliferation, cell division, cell growth, the cell cycle, cell differentiation, cell migration, axonogenesis, nerve cell interactions, angiogenesis, and regeneration. Signaling pathways mediated by receptor tyrosine kinases may be initiated by growth factors binding to specific receptors on cell surfaces. One such growth factor is epidermal growth factor (EGF) which induces proliferation of a variety of cells in vivo. The binding of EGF to its receptor (epidermal growth factor receptor—EGFR) activates a RTK/PTK signaling pathway. The EGF receptor has an extracellular N-terminal domain that binds EGF and a cytoplasmic C-terminal domain containing an EGF-dependent protein tyrosine kinase that is capable of autophosphorylation and the phosphorylation of other protein substrates. The binding of EGF to its receptor activates the tyrosine kinase which phosphorylates a variety of signaling molecules thereby initiating a RTK/PTK signaling pathway that leads to DNA replication, RNA and protein synthesis, and cell division. Other RTK/PTK signaling pathways can be activated through the following receptor tyrosine kinases: PDGFR, insulin receptor tyrosine kinase, Met receptor tyrosine kinase, fibroblast growth factor (FGF) receptor, insulin receptor, insulin growth factor (IGF-1) receptor, TrkA, B, C receptors, TIE-1, Tek/Tie2, Flt-1, Flk, VEGFR3, EFGR/ErbB, ErbB2/neu, ErbB3, Ret, Kit, Alk, Axl, FGFR1, FGFR2, FGFR3, keratinocyte growth factor (KGF) receptor, EphA receptors including but not limited to EphA1 (also known as Eph and Esk), EphA2 (also known as Eck, Myk2, Sek2), EphA3 (also known as Cek4, Mek4, Hek, Tyro4, Hek4), EphA4 (also known as Sek, Sek1, Cek8, Hek8, Tyrol), EphA5 (also known as Ehk1, Bsk, Cek7, Hek7, and Rek7), EphA6 (Ehk2, and Hek12) EphA7 (also known as Mdk1, Hek11, Ehk3, Ebk, Cek11), and EphA8 (also known as Eek, Hek3); and the Eph B receptors including but not limited to EphBI (also known as Elk, Cek6, Net, Hek6), EphB2 (also known as Cek5, Nuk, Erk, Qek5, Tyro5, Sek3, hek5, Drt), EphB3 (also known as Cek10, Hek2, Mdk5, Tyro6, and Sek4), EphB4 (also known as Htk, Myk1, Tyro11, Mdk2), EphB5 (also known as Cek9, Hek9), and EphB6 (also known as Mep).

Protein tyrosine kinases (i.e. intracellular tyrosine kinases) that activate RTK/PTK signaling pathways include members of the Src family including Src, Fyn, Yes, Lyn, Lck, Yrk, Hrk, and Blk; members of the BTK family including BTK, Tec, and Itk; members of the Jak family including Jak1, Jak2, and Jak3; and Abl, Fak, Zap70, Syk, Tyk, Fer, Fes, Csk, Ntk, Pyk. [See Qui Y and Kung H J, Oncogene 2000 Nov. 20;19(49):5651-61; Hubbard SR and Till J H., Annu Rev Biochem 2000;69:373-98; Tatosyan A G, and Mizenina O A. Biochemistry (Mosc) 2000 January;65(1):49-58).]

An RTK/PTK signaling pathway that mediates cell proliferation is referred to herein as a “mitogenic signaling pathway”. A RTK/PTK signaling pathway that results in uncontrolled growth of cells (i.e. cancer cells) is referred to as an “oncogenic signaling pathway”.

“RTK/PTK signal” refers to the stimuli that activate a RTK/PTK signaling pathway resulting in cell proliferation. A RTK/PTK signal may be a growth factor. An RTK/PTK signal that activates an oncogenic signaling pathway resulting in uncontrolled growth of cells is referred to herein as an “oncogenic signal”.

“A domain of a signaling molecule that regulates a RTK/PTK signal” refers to a modular structural element that transduces a signal in a RTK/PTK signaling pathway. Such a domain is also referred to herein as a “RTK/PTK signal domain”. In some aspects of the invention the term includes proteins or agents containing the domain. The domain may be from any species, particularly a mammalian species, including bovine, ovine, porcine, murine, equine, preferably the human species, and from any source, whether natural, synthetic, semi-synthetic, or recombinant. Examples of RTK/PTK signal domains include modular structural elements that recognize phosphotyrosine-containing sites on proteins (e.g. receptor protein tyrosine kinases) including but not limited to Src-homology-2 (SH2) domains and pTyr-binding (PTB) domains.

“SH2 domains” are noncatalytic domains of about 100 amino acids (Pawson, T. Oncogene 3: 491, 1988). The domain comprises five-well conserved sequence motifs that are separated by more variable sequence elements. The variable regions generally contain one or more glycine or proline residues. SH2 domains bind phosphotyrosine-containing polypeptides via 2 surface pockets. Specificity is provided via interaction with residues that are distinct from the phosphotyrosines. An SH2 domain that may be used in a molecule, chimeric protein, or method of the invention includes an SH2 domain of Grb2, Src, Yes, Fgr, Fyn, Lck, Lyn. Hck, Blk, Abl, Fps, Per, PLC-γ, Gap, v-Crk, p85, Nck, Vav, ZAP 70, and tensin, and homologues of these proteins. For reviews discussing SH2 domains see Pawson, 1995, Nature 373:573-580; Cohen et al., 1995, Cell 80:237-248; Pawson and Gish, 1992, Cell 71:359-362; and Koch et al., 1991, Science 252:668-674. Also see Simple Modular Architecture Research Tool (SMART) at http://smart.embl-heidelberg.de for references describing SH2 domains and sequences of SH2 domains.

A “PTB” domain is a region of ˜160 amino acids which was originally identified in Shc and Sck (Kavanaugh, V. M. Et al., 1995 Science, 268:1177-1179; Bork, RP, and Margolis, B, Cell, Vol 80:693-694, 1995; Craparo, A., et al., 1995, J. Biol. Chem. 270:15639-15643; van der Geer, P., & Pawson, T., 1995, TIBS 20:277-280; Batzer, A. G., et al., Mol. Cell. Biol. 1995, 15:4403-4409; and Trub, T., et al., 1995, J. Biol. Chem. 270:18205-18208; van der Geer et al., Current Biology 5(4):404, 1995)). The PTB domain comprises residues 46 to 208 in the 52 kDa isoform of Shc. (See Simple Modular Architecture Research Tool (SMART) at http://smart.embl-heidelberg.de for references describing PTB domains and sequences of PTB domains). A PTB domain that may be used in a molecule, chimeric protein, or method of the invention includes a PTB domain of Shc, Sck, IRS-1, IRS-2, and NUMB and homologues of these proteins.

“Homologue” refers to a protein with sequence identity or similarity to a selected protein. The term “similarity” refers to a degree of complementary. There may be partial similarity, substantial similarity, or complete similarity. The word “identity” in some cases may substitute for the word “similarity”. Both identity and similarity can be readily calculated (Computational Molecular Biology, Lesk, A. M., ed., Oxford University Press New York, 1988; Biocomputing: Informatics and Genome Projects, Smith, D. W. ed., Academic Press, New York, 1993; Computer Analysis of Sequence Data, Part I, Griffin, A. M., and Griffin, H. G. eds. Humana Press, New Jersey, 1994; Sequence Analysis in Molecular Biology, von Heinje, G., Academic Press, New York, 1987; and Sequence Analysis Primer, Gribskov, M. and Devereux, J., eds. M. Stockton Press, New York, 1991).

A partially complementary sequence that at least partially inhibits hybridization of an identical sequence to a target nucleic acid is referred to as “substantially similar”. Hybridization assays (e.g. Southern or northern blots, solution hybridization) can be used to examine inhibition of hybridization of a completely complementary sequence to the target sequence. A sequence that is substantially similar will compete for and inhibit the binding of a completely similar (i.e. identical) sequence to the target sequence under conditions that require that the binding of two sequences to one another be a selective interaction (e.g. reduced stringency).

Proteins that contain homologous SH2, PTB, death, or death effector domains may be identified using data base search methods (Bork, R P, and Margolis, B. Cell, Vol 80:693-694, 1995). Other methods may be utilized such as generalized profile techniques and Hidden Markov Model searches (Hoffman, supra and references therein). Proteins that contain SH2, PTB, death, or death effector domains may also be identified by screening a cDNA expression library with a protein containing a sequence with high affinity to an SH2 domain, PTB domain, death, or death effector domain. Proteins that contain SH2, PTB, or death effector domains may also be screened using antibodies specific for the domains. One could use PCR (Wilks, A. F., Proc. Natl. Acad. Sci. U.S.A. Vol. 86, pp. 1603-1607, March 1989) or low stringency screening (Hanks, S. K., Proc. Natl. Acad. Sci. U.S.A. Vol. 84, pp 388-392, January 1987) with an SH2, PTB, death, or death effector domain specific probe.

“Signaling molecule” refers to a molecule that transduces signals (e.g. apoptotic signal, and RTK/PTK signal) in a signaling pathway including an apoptotic signaling pathway and RTK/PTK signaling pathway. Signaling molecules transduce signals by interacting with one another through specific domains (e.g. RTK/PTK signal domain or apoptotic domain) that mediate the recognition of one molecule by another.

“Ligand-binding domain” refers to a domain which allows for binding to a natural or unnatural oligomerizing ligand which mediates dimerization or oligomerization of chimeric proteins of the invention. In an embodiment of the invention the domain induces formation of dimers between a chimeric protein comprising a domain of a signaling molecule that regulates an RTK/PTK signal and a ligand-binding domain, and a chimeric protein comprising a domain of a signaling molecule that promotes apoptosis in the cell and a ligand-binding domain.

Oligomerizing ligands may be mulitvalent, preferably cell permeant, compounds, generally having a molecular weight below about 5 kD, preferably below 2 kD, which mediate formation of complexes with proteins containing ligand-binding domains to which the ligand binds. Examples of oligomerizing ligands include FK506, FK1012, rapamycin, cyclosporin A, coumermycin, fujisporin, and analogs thereof, and other synthetic dimerizers. Ligand-binding domains include the FK506 binding domain of FKBP, the cyclosporin-binding domain of calcineurin, the rapamycin-binding domain of FRAP, the coumermycin binding domain of DNA Gyrase, the fujisporin binding domain of cyclophilin or FKBP, and the rapamycin binding domain of FKBP. (See U.S. Pat. No. 5,994,313 for examples of dimerization methods, and PCT/US93/01617 for a discussion of binding domains and ligands.)

Molecules and Chimeric Proteins of the Invention

The invention contemplates a molecule comprising a domain of a signaling molecule that regulates a RTK/PTK signal in a cell and a domain of a signaling molecule that promotes apoptosis in a cell. In an embodiment of the invention, the domain that regulates a RTK/PTK signal is a domain that binds to a phosphotyrosine-containing protein, preferably an SH2 domain or a PTB domain, and the domain that regulates an apoptotic signal is a death effector domain, preferably a death effector domain of an adaptor protein. A molecule comprising a death effector domain and SH2 domain is referred to herein as “DED-SH2”; and a molecule comprising a death effector domain and a PTB domain is referred to herein as “DED-PTB”.

In preferred embodiments the molecule comprises a Grb2 SH2 domain or a PTB domain of Shc, and the death effector domain of FADD. In preferred embodiments, the molecule is a DED-SH2 having the sequence shown in FIG. 1A, or a DED-PTB having the sequence shown in FIG. 1B.

The invention also contemplates chimeric proteins. In an embodiment, a chimeric protein is provided comprising a domain of a signaling molecule that regulates a RTK/PTK signal in a cell and a ligand-binding domain for binding to an oligomerizing ligand. A chimeric protein is also provided comprising a signaling molecule that promotes apoptosis in the cell and a ligand-binding domain for binding to an oligomerizing ligand. In a preferred embodiment, the ligand-binding domain is the FK506 binding domain of FKBP, or the rapamycin-binding domain of FRAP.

The invention also provides an oligomer comprising two or more chimeric proteins of the invention complexed through an oligomerizing ligand. In an embodiment, a dimer is provided comprising a first chimeric protein complexed to a second chimeric protein via an oligomerizing ligand wherein the first chimeric protein comprises a domain of a signaling molecule that regulates a RTK/PTK signal in a cell and a ligand-binding domain that binds to the oligomerizing ligand; and the second chimeric protein comprises a signaling molecule that promotes apoptosis in the cell and a ligand-binding domain that binds to the oligomerizing ligand. In particular embodiments the oligomerizing ligand is FK506 or rapamycin and the ligand-binding domain is the FK506 binding domain of FKBP or the rapamycin-binding domain of FRAP, respectively.

A molecule or chimeric protein of the invention may be conjugated with other molecules, such as proteins, to prepare fusion proteins. This may be accomplished, for example, by the synthesis of N-terminal or C-terminal fusion proteins.

A molecule, or chimeric protein comprising a signaling molecule that promotes apoptosis, may be complexed with a caspase. Thus, the invention relates to a complex comprising a molecule, or chimeric protein comprising a signaling molecule that promotes apoptosis, in association or complexed with a caspase. In an embodiment, the complex comprises caspase 8 or caspase 9.

A molecule, chimeric protein, or complex of the invention may be tagged with a substance that targets the molecule, chimeric protein, or complex to a particular cell type or tissue. For example, a molecule, chimeric protein, or complex of the invention may be tagged with a cell surface ligand or an antibody against cell surface antigens of a target tissue (e.g. tumor antigens).

The invention also contemplates antibodies specific for molecules, chimeric proteins, or complexes of the invention. The antibodies may be intact monoclonal or polyclonal antibodies, and immunologically active fragments (e.g. a Fab or (Fab)₂ fragment), an antibody heavy chain, and antibody light chain, a genetically engineered single chain Fv molecule (Ladner et al, U.S. Pat. No. 4,946,778), humanized antibodies, or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

A molecule, chimeric protein, or complex of the invention may be prepared using recombinant DNA methods. Accordingly, nucleic acid molecules which encode a molecule, chimeric protein, or complex of the invention may be incorporated in a known manner into an appropriate expression vector which ensures good expression of the molecule, chimeric protein, or complex. Possible expression vectors include but are not limited to cosmids, plasmids, or modified viruses so long as the vector is compatible with the host cell used. The expression vectors contain a nucleic acid molecule encoding a molecule, chimeric protein, or complex of the invention and the necessary regulatory sequences for the transcription and translation of the inserted sequence. Suitable regulatory sequences may be obtained from a variety of sources, including bacterial, fungal, viral, mammalian, or insect genes. (For example, see the regulatory sequences described in Goeddel, Gene Expression Technology: Methods in Enzymology 185, Academic Press, San Diego, Calif. (1990)). Selection of appropriate regulatory sequences is dependent on the host cell chosen, and may be readily accomplished by one of ordinary skill in the art. Other sequences, such as an origin of replication, additional DNA restriction sites, enhancers, and sequences conferring inducibility of transcription may also be incorporated into the expression vector.

The recombinant expression vectors may also contain a selectable marker gene which facilitates the selection of transformed or transfected host cells. Suitable selectable marker genes are genes encoding proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

The recombinant expression vectors may also contain genes that encode a fusion portion which provides increased expression of the recombinant molecule; increased solubility of the recombinant molecule; and/or aid in the purification of the recombinant molecule by acting as a ligand in affinity purification. For example, a proteolytic cleavage site may be inserted in the recombinant molecule to allow separation of the recombinant molecule from the fusion portion after purification of the fusion protein. Examples of fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding protein, or protein A, respectively, to the recombinant protein. The recombinant expression vector may include genes that cause expression of a molecule, chimeric protein, or complex of the invention in a specific cell type or tissue.

Recombinant expression vectors may be introduced into host cells to produce a transformant host cell. Transformant host cells include prokaryotic and eukaryotic cells that have been transformed or transfected with a recombinant expression vector of the invention. The terms “transformed with”, “transfected with”, “transformation” and “transfection” are intended to include the introduction of nucleic acid (e.g. a vector) into a cell by one of many techniques known in the art. For example, prokaryotic cells can be transformed with nucleic acid by electroporation or calcium-chloride mediated transformation. Nucleic acid can be introduced into mammalian cells using conventional techniques such as calcium phosphate or calcium chloride co-precipitation, DEAE-dextran-mediated transfection, lipofectin, electroporation or microinjection. Suitable methods for transforming and transfecting host cells may be found in Sambrook et al. (Molecular Cloning: A Laboratory Manual, 2nd Edition, Cold Spring Harbor Laboratory press (1989)), and other laboratory textbooks.

Suitable host cells include a wide variety of prokaryotic and eukaryotic host cells. For example, the peptides of the invention may be expressed in bacterial cells such as E. coli, insect cells (using baculovirus), yeast cells or mammalian cells (e.g. Cos 1, Hela, and NIH 3T3). Other suitable host cells can be found in Goeddel, Gene Expression Technology: Methods in Enzymology 185, Academic Press, San Diego, Calif. (1991).

The molecules, chimeric proteins, and complexes of the invention may be synthesized by conventional techniques. For example, they may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford III. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles fo Peptide Synthesis, Springer-Verlag, Berlin 1984, and E. Gross and J. Meienhofer, Eds., The Peptides: Analysis, Synthesis, Biologu, suprs, Vol 1, for classical solution synthesis).

N-terminal or C-terminal fusion proteins comprising a molecule or chimeric protein of the invention conjugated with other molecules (e.g. a complex of the invention) may be prepared by fusing, through recombinant techniques, the N-terminal or C-terminal of the peptide, and the sequence of a selected protein or selectable marker with a desired biological function. The resultant fusion proteins contain the peptide fused to the selected protein or portion thereof, or marker protein as described herein. Examples of proteins which may be used to prepare fusion proteins include cell surface ligands, immunoglobulins, glutathione-S-transferase (GST), hemagglutinin (HA), and truncated myc.

Cyclic derivatives of the molecules or chimeric proteins of the invention are also part of the present invention. Cyclization may allow the molecules or proteins to assume a more favorable conformation for association with a signaling molecule in a RTK/PTK signaling pathway or an apoptotic signaling pathway. Cyclization may be achieved using techniques known in the art.

The molecules, chimeric proteins, and complexes of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

Computer modelling techniques known in the art may be used to observe the interaction of a molecule, oligomerized chimeric protein, or complex of the invention with a selected signaling molecule in a RTK/PTK signaling pathway or an apoptotic signaling pathway (for example, Homology Insight II and Discovery available from BioSym/Molecular Simulations, San Diego, Calif., U.S.A.). If computer modeling indicates a strong interaction, the molecule can be synthesized and tested for its ability to modify a RTK/PTK signal in a cell as discussed herein. The interactions may also be characterized using the methods described herein (e.g. in the Examples).

Compositions and Methods

An apoptotic domain, RTK/PTK signal domain, molecules, chimeric protein, oligomer, and complex of the invention may be formulated into pharmaceutical compositions. The compositions can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of one or both domains, molecules, or chimeric proteins, in association with one or more pharmaceutically acceptable vehicles or diluents, and contained in buffered solutions with a suitable pH and iso-osmotic with the physiological fluids.

An apoptotic domain, RTK/PTK signal domain, molecule, chimeric protein, oligomer, or complex of the invention can be in a composition which aids in delivery into the cytosol of a cell. The substance may be conjugated with a carrier moiety such as a liposome that is capable of delivering the substance into the cytosol of a cell (See for example Amselem et al., Chem. Phys. Lipids 64:219-237, 1993 which is incorporated by reference). Alternatively, a substance may be modified to include specific transit peptides or fused to such transit peptides that are capable of delivering the substance into a cell. The substances can also be delivered directly into a cell by microinjection.

An apoptotic domain, RTK/PTK signal domain, molecule, chimeric protein, or complex of the invention may be therapeutically administered by implanting into a subject, vectors or cells capable of producing the domain, molecule, chimeric protein, or complex. In one approach cells that secrete a domain, molecule, chimeric protein, or complex may be encapsulated into semipermeable membranes for implantation into a subject. The cells can be cells that have been engineered to express a domain, molecule, chimeric protein, or complex. It is preferred that the cell be of human origin and the domain, molecule, chimeric protein, or complex be derived from a human domain, molecule, protein, or complex when the subject is a human.

The molecules, compositions, oligomers, complexes, and domains described herein are for administration to subjects in a biologically compatible form suitable for administration in vivo. By “biologically compatible form suitable for administration in vivo” is meant a form of the substance to be administered in which any toxic effects are outweighed by the therapeutic effects.

The substances may be administered to living organisms including humans, and animals (e.g. dogs, cats, cows, sheep, horses, rabbits, and monkeys). Preferably the substances are administered to human and veterinary patients.

Administration of a “therapeutically active amount” is defined as an amount of a substance, at dosages and for periods of time necessary to achieve the desired result. For example, a therapeutically active amount of a substance may vary according to factors such as the disease state, age, sex, and weight of the individual, and the ability of the substance to elicit a desired response in the individual. Dosage regima may be adjusted to provide the optimum therapeutic response. For example, several divided doses may be administered daily or the dose may be proportionally reduced as indicated by the exigencies of the therapeutic situation. A therapeutically active amount can be estimated initially either in cell culture assays e.g. of neoplastic cells, or in animal models such as mice, rats, rabbits, dogs, or pigs. Animal models may be used to determine the appropriate concentration range and route of administration for administration to humans.

The active substance may be administered in a convenient manner by any of a number of routes including but not limited to oral, subcutaneous, intravenous, intraperitoneal, intranasal, enteral, topical, sublingual, intramuscular, intra-arterial, intramedullary, intrathecal, inhalation, transdermal, or rectal means. The active substance may also be administered to cells in ex vivo treatment protocols. Depending on the route of administration, the active substance may be coated in a material to protect the substance from the action of enzymes, acids and other natural conditions that may inactivate the substance.

The nucleic acid molecules encoding a molecule, chimeric protein, or complex of the invention may be used for therapeutic purposes. Viral gene delivery systems may be derived from retroviruses, adenoviruses, herpes or vaccinia viruses or from various bacterial plasmids for delivery of nucleic acid sequences to the target organ, tissue, or cells. Vectors that express the molecules, chimeric proteins, or complexes can be constructed using techniques well known to those skilled in the art (see for example, Sambrook et al.). Non-viral methods can also be used to cause expression of a molecule, chimeric protein, or complex of the invention in tissues or cells of a subject. Most non-viral methods of gene transfer rely on normal mechanisms used by mammalian cells for the uptake and transport of macromolecules. Examples of non-viral delivery methods include liposomal derived systems, poly-lysine conjugates, and artificial viral envelopes.

In viral delivery methods, vectors may be administered to a subject by injection, e.g. intravascularly or intramuscularly, by inhalation, or other parenteral modes. Non-viral delivery methods include administration of the nucleic acid molecules using complexes with liposomes or by injection; a catheter or biolistics may also be used.

An oligomerizing ligand may be administered to a subject to oligomerize (e.g. dimerize) chimeric proteins of the invention. The ligand may activate transcription of a nucleic acid encoding a chimeric protein of the invention. Various protocols may be employed depending upon the binding affinity of the ligand, the response desired, the manner of administration, the half-life, and the number of cells present. An oligomerizing ligand may be formulated using conventional methods and materials well known in the art. The dose and method of administration will depend on the above factors and it will be determined by the attending healthcare provider. In an embodiment of the invention, the ligand is administered parenterally or orally. Activation by an oligomerizing ligand may be terminated by administering a monomeric compound which can compete with the administered ligand, i.e. an antagonist of the ligand.

The therapeutic efficacy and safety of the molecules, chimeric proteins, complexes, nucleic acids, and compositions of the invention can be determined by standard pharmaceutical procedures in cell cultures or animal models.

Antibodies that specifically bind the therapeutically active ingredient may be used to measure the amount of the therapeutic active ingredient in a sample taken from a patient for the purposes of monitoring the course of therapy.

Applications

The apoptotic and RTK/PTK signal domains, molecules, nucleic acids, oligomers, complexes, and compositions of the invention may be used to induce apoptosis in cells. Induction of apoptosis using apoptotic and RTK/PTK signal domains, and the molecules, nucleic acids, oligomers, complexes, and compositions of the invention can be used to treat or prevent disease conditions where the affected cells have a defective RTK or PTK (i.e. mutated RTK or PTK or over expressed RTK or PTFK) or a defective ligand for the RTK or PTK (e.g. mutated or over expressed ligand) resulting in, for example, an up-regulation of cell growth. Table 1 provides examples of RTKs involved in cancer (e.g. activating mutation or overexpression). In an aspect of the invention the disease condition is associated with a defective EGFR or PDGFR.

The present invention may be particularly useful in treating or preventing diseases associated with increased cell survival, or the inhibition of apoptosis, include cancers (e.g. follicular lymphomas, carcinomas with p53 mutations, hormone-dependent tumors such as breast cancer, prostate cancer, Kaposi's sarcoma and ovarian cancer); autoimmune disorders (such as lupus erythematosus and immune-related glomerulonephritis rheumatoid arthritis) and viral infections (such as herpes viruses, pox viruses, and adenoviruses); inflammation, graft vs. host disease, acute graft rejection and chronic graft rejection.

In particular, the molecules, chimeric proteins, complexes, oligomers, nucleic acids, compositions and methods of the invention may be used to prevent or treat lymphoproliferative conditions, malignant and pre-malignant conditions, arthritis, inflammation, vasculogenesis/angiogenesis, neurological disorders, and autoimmune disorders. Malignant and pre-malignant conditions may include solid tumors, B cell lymphomas, chronic lymphocytic leukemia, chronic myelogenous leukemia, prostate hypertrophy, Hirschsprung disease, glioblastoma, breast and ovarian cancer, adenocarcinoma of the salivary gland, promyelocytic leukemia, prostate cancer, multiple endocrine neoplasia type IIA and IIB, medullary thyroid carcinoma, papillary carcinoma, papillary renal carcinoma, hepatocellular carcinoma, gastrointestinal stromal tumors, sporadic mastocytosis, acute myeloid leukemia, large cell lymphoma or Alk lymphoma, melanoma, chronic myeloid leukemia, hematological/solid tumors, papillary thyroid carcinoma, stem cell leukemia/lymphoma syndrome, acute myelogenous leukemia, osteosarcoma, multiple myeloma, preneoplastic liver foci, and resistance to chemotherapy.

The following non-limiting examples are illustrative of the present invention:

### EXAMPLE 1

FADD is comprised of a death domain and a DED domain. The death domain of FADD binds to the death domains of ligand bound death receptors. The DED domain of FADD binds to DED or the caspase recruitment domain (CARD) of initiator caspase 8 or 10. Caspases are cysteine proteases that normally exist in inactive states, called procaspases. Recruitment of the caspase 8 to the activated death receptor, clusters the enzyme, thereby activating it, and initiating apoptosis. It has been surprisingly shown that clustering of caspase is achieved through growth factor induced clustering of RTK or PTK. The growth factor induces oligomerization of the RTK or PTK receptor and leads to activation of the kinase domain, auto-phosphorylation, and recruitment of DEDSH2 and associated caspase. Recruitment to the kinase complex of multiple molecules of DEDSH2 and bound caspase increases the local concentration of caspase thereby stimulating its activation, and initiation of apoptosis. Thus, for the first time it has been shown that growth factor signaling can be directly coupled to apoptosis. The utility of this discovery will be in the application to cancer treatment. Many cancers over express RTKs or PTKs, such as the EGFR or PDGFR. SH2 and PTB domains of molecules like GRB2 and SHC interact directly with activated forms of both RTKs and PTKs and are responsible for mediating the signaling from them. Therefore, expression of DED-SH2 or DED-PTB molecules in tumors can be used to initiate apoptosis and killing of cancer cells. It should be possible to utilize the modular nature of many signaling proteins to redesign or engineer pathways for cancer treatment, either through drug design to artificially couple molecules such as GRB2 and FADD, or through gene therapy to introduce DED-SH2 or DED-PTB like genes into cancer cells.

The present inventors utilized the innate suicide mechanism of cells against cancer cells by creating a new pathway which links RTK signaling to apoptosis. Instead of activating uncontrolled cell growth in cancerous cells, the new pathway initiates apoptosis. The modular feature of proteins was used to construct novel molecules, referred to as “DED-SH2” (Death Effector Domain-Src Homology 2 domain) or DED-PTB (Death Effector Domain-Phospho-Tyrosine Binding Domain). These molecules provide a novel nexus between receptor tyrosine kinases and the apoptosis pathway. For these molecules, modular domains were used from three different adaptor molecules: two from the RTK pathway and the other from the apoptosis pathway. In particular, the SH2 domain of Grb2 was combined with a death effector domain (DED) of another adaptor protein, called FADD, and the PTB domain of Shc A was combined with a death effector domain (DED) of FADD.

Materials and Methods

Constructs:

The cDNA encoding the death effector domain (DED) of Fadd was amplified from randomly primed complementary DNA (cDNA) prepared from 1 μg of total RNA isolated from the C₂C₁₂ myogenic cell line. The cDNA (2 μl) was used as a template for nested polymerase chain reaction (PCR) amplification. An initial PCR amplification was performed using primers dedf1 (5′-cgagatttacccatggac-3′) [SEQ ID NO. 5] and dedr1 (5′-gtcaaatgccacctgcag-3′) [SEQ ID NO. 6] for 25 cycles of 94° C. for 30 seconds, 60° C. for 1 minute, and 68° C. for 3 minutes in a Perkin Elmer Cetus 480 themal cycler. The product from this reaction (1 μl) was then used as a template for a nested PCR amplification using primers dedf2 (5′-ttaagcttatggacccattcctggtgctgc-3′)) [SEQ ID NO. 7] and dedr2 (5′-agaattctcgaagtcgtccaggcgctgcag-3′)) [SEQ ID NO. 8] under the same cycling conditions used above. The amplified product was purified, digested with Xba-I and EcoRI, and ligated into the Xba-1/EcoRI sites of pcDNA 3.

The SH2 domain of Grb2 was PCR amplified from a plasmid containing full length human Grb2 using primers SH2f1(5′-aagaattcgtggttttttggcaaaatcc-3′)) [SEQ ID NO. 9] and SH2 r1(5′-aatctagactgttgtatgtcccgtaag-3′)) [SEQ ID NO. 10] under the same cycling conditions used above. The SH2 cDNA was purified, digested with EcoRI and HindIII and ligated into the EcoRI/HindIII sites in pcDNA3 containing the fadd DED cDNA. All constructs were verified by restriction digest analysis and sequencing. (See FIG. 1A). Expression of full-length fusion proteins was confirmed by Western analysis following transfection of Cos 1 cells.

Cell Culture:

Cells were grown in DME (gentamycin 20 μg per ml, and 2×glucose) supplemented with 10% calf serum. Prior to transduction, cells were split and grown to approximately 60% confluency. Cells were infected with either adenoviruses DEDSH2mychis, DEDSH2 (R86K) mychis, or SH2mychis. Adenoviruses were prepared according to the method of Prevec and Graham (Mol Biotechnol 1995 June;3(3):207-20). Cells were then serum starved overnight, then cells were stimulated with epidermal growth factor (EGF 100 ng/ml) in serum free media containing 20 mm Hepes (pH 7.4). The cells were left for 24 hours before analysis.

Immunoprecipitation:

PLC Lysates were cleared by centrifugation and 3000 rpm at 4° C. To preclear the lysates, the supernatants were incubated with 100 μl of 10% antimouse antibody conjugated to sepharose beads for 1 hour. The lysates were then incubated with 3 μg/ml anti-cmyc for one hour at 4° C. ErbB2/DEDSH2/antibody complexes were precipitated using 100 μl of 10% of antimouse antibody conjugated to sepharose beads. The beads were resuspended in 50 μl of 1×SDS sample buffer (25 mmol/L tris, 1% SDS, 50 mmol/dithiothreitol, 5% glycerol, 0.1% bromophenol blue), and boiled for 3 minutes. The supernatant was collected and separated by SDS-PAGE, and electroblotted to PVDF membrane for Western probing.

Apoptosis Assays:

Cells transduced with DEDSH2 or DEDSH2(R86K) or SH2 adenoviruses were serum starved and then stimulated with either 10% FBS or EGF (100 ng/ml) for 24 hours, before measuring apoptosis. Four standard assays were used to determine apoptosis levels caspase activity, DNA laddering, cell survival staining (crystal violet), and PARP cleavage.

Western Analysis

Cells were washed with PBS and lysed in PLC lysis buffer for protein extraction. Blots were blocked overnight in TBST (10 mmol/L Tris pH 8.0, 150 mmol/L NaCl, and 0.05% Tween-20) with 5% low-fat, and incubated with primary antibodies (0.5 μg/ml) in TBST with 1% low-fat milk for 1 hour. Blots were washed with TBST for 10 minutes×3. Secondary antimouse immunoglobulin (Ig) G conjugated to horse radish peroxidase (dilution 1:10 000) in TBST with 1% low-fat milk was applied to the blots and incubated for 1 hour. Blots were washed in TBST and processed by Enhanced Chemical Luminescence.

Results

The results are shown in the Figures.

### EXAMPLE 2

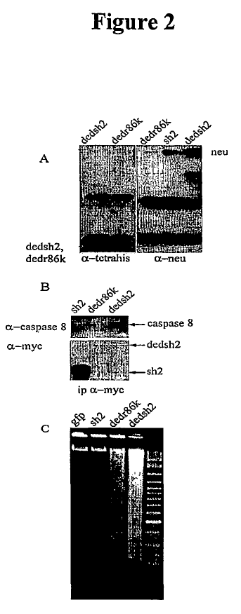

Expression of DEDSH2 adaptors should lead to formation of unique death inducing signaling complexes (disc) in expressing cells. Specifically, expression of DEDSH2 should lead to recruitment of DEDSH2 and caspase 8 to activated receptor tyrosine kinases (RTKs). To test this, protein interactions were studied between death adaptors, caspase 8, and NeuNt, a constitutively active form of the RTK, ERB-B2. Ntr2 cells (NeuNt transformed cell line derived from Rat2 cells) were incubated overnight with DEDSH2, DEDR86K, or SH2 bearing adenoviruses. Adaptor molecules were immunoprecipitated with anti-myc antibody and blotted with either tetra-his antibody, or ERB-B2 (Neu) antibody. As expected, NeuNt co-precipitated with DEDSH2 and SH2 (FIG. 2A). A weak band in DEDR86K lysates was also detected. This is consistent with results by Rameh et al (Cell. 1995 Dec. 1;83(5):821-30) on the p85 subunit of PI 3 kinase, indicating that mutation of the arginine to lysine within the FLVR sequence greatly reduces but does not abolish SH2 domain function. Immunoprecipitates were also examined for the presence of caspase 8. As shown in FIG. 2B, caspase 8 co-immunoprecipitates with DEDSH2 and DEDR86K, but not with SH2 domain alone.

Apoptosis is characterized by the activation of a caspase cascade that leads to the cleavage of nuclear DNA into small fragments that produce a characteristic laddering effect when examined by agarose gel electrophoresis. This feature distinguishes apoptotic cell death from necrotic death. To confirm the NeuNt transformed cells were dying of apoptosis and not necrosis, DNA from infected Ntr2 cells was examined. Expression of DEDSH2, or DEDR86K, but not SH2 or GFP, in Ntr2 cells, produces DNA laddering characteristic of apoptosis. As expected the laddering was more pronounced in DEDSH2 cells than in DEDR86K cells (FIG. 2C).

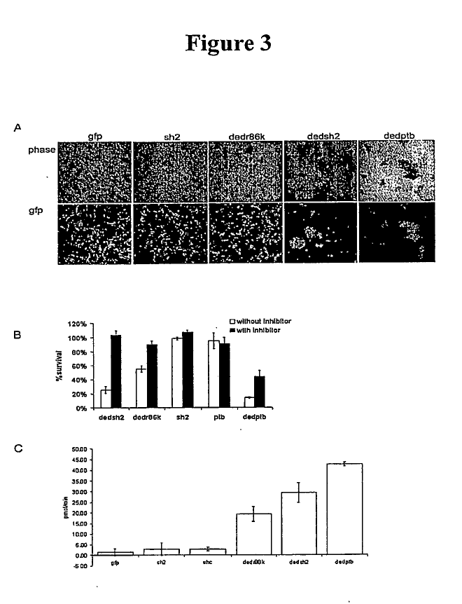

FIG. 3A shows the appearance of Ntr2 cells 24 hours after treatment with DEDSH2, DEDR86K, SH2, PTB, or DEDPTB bearing adenoviruses. Treatment of the transformed cells with DEDSH2 or DEDPTB resulted in their death, and the appearance of apoptotic bodies, which appear as round floating spheres. The lower panel shows the corresponding GFP fluorescence and indicates that all of the cells were infected with virus. Recruitment of caspase 8-DEDSH2 or caspase 8-DEDPTB complexes to activated RTKs should lead to caspase 8 activation and apoptosis. To determine whether the cell death observed upon treatment of Ntr2 cells with DEDSH2 or DEDPTB was due to caspase activation, cell survival was measured in the presence or absence of caspase inhibitor (FIG. 3B). Co-treatment of Ntr2 cells with a caspase inhibitor restored cell viability to DEDSH2 and DEDPTB expressing cells. DEDSH2 adaptors are able to interact with caspase 8 and NeuNt by immunoprecipitation. Whether recruitment of caspase 8 to NeuNt by DEDSH2 leads to caspase 8 activation was tested next. The ability of lysates of treated cells to cleave a synthetic caspase 8 substrate, IETD-p-nitroalanine was tested. As shown in FIG. 3C, substantial caspase 8 activity was observed in lysates from DEDPTB, DEDSH2, and DEDR86K expressing cells. Strongest activation was achieved by DEDPTB expression, followed by DEDSH2 and DEDR86K, respectively. This coincides with the cell survival assay and DNA laddering data. Taken together, these data support the conclusion that recruitment of caspases to activated RTKs by DEDSH2, and DEDPTB adaptors can be used to induce apoptosis and kill transformed cells.

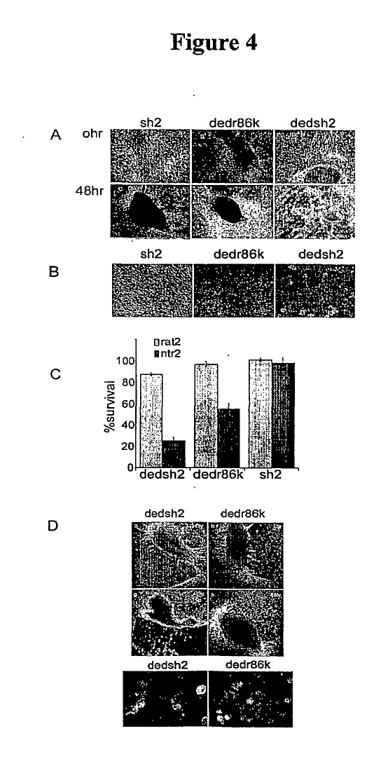

Whether the cell killing observed was specific for transformed cells was tested. Non transformed Rat 2 cells were transfected with NeuNt to generate a population of cells in which transformed cells (NeuNt transfected) are mixed with non transformed cells (untransfected). Under these conditions, Rat 2 cells that have been transfected with NeuNt, lose contact inhibition and develop into foci; whereas, untransfected cells, which remain contact inhibited, surround the foci in a monolayer. The cells were then incubated with adenoviruses bearing DEDSH2, SH2, or DEDR86K, and individual foci were examined by phase contrast microscopy. As shown in FIG. 4A, foci treated with DEDSH2, were killed after 48 hours. In contrast, foci treated with SH2 or DEDR86K remained intact. Surprisingly, the surrounding monolayer of non-transformed Rat 2 cells survived treatment with DEDSH2 (FIG. 4B), indicating they are less sensitive to apoptosis induced by this method. GFP fluorescence in the monolayer and foci confirmed that both the transformed and nontransformed cells were transduced with virus. The selectivity of killing for transformed cells was unexpected since RTKs also provide important survival and differentiation signals to cells. It suggests that induction of apoptosis by DEDSH2 adaptors is dependent on the deregulated signal produced by oncogenic NeuNt, and that regulated RTK signaling in quiescent cells is insufficient to induce apoptosis. The difference in sensitivity was measured by comparing the survival of parental Rat 2 to Ntr2 (NeuNt transformed cell line) cells. As shown in FIG. 4C, NeuNt transformed cells expressing DEDSH2 showed reduced survival when compared to the parental cell line. To rule out spurious contribution of adenoviral proteins to the apoptosis observed with DEDSH2, fusion proteins of DEDSH2 and DEDR86K, were also generated in which the adaptors were fused to a mps (membrane permeable sequence) from the HIV tat protein. This sequence from tat allows fusion proteins to transfer across biological membranes. FIG. 4D shows foci derived from transfection of Rat 2 cells with NeuNt, and treated with fusion proteins of TAT-DEDSH2, or TAT-DEDR86K. Foci from TAT-DEDSH2 treated cells underwent apoptosis; whereas DEDR86K treated cells remained unaffected, indicating that the apoptosis in NeuNt transformed cells was specific for DEDSH2 and not dependent upon adenoviral proteins. The bottom panel shows immunofluorescence (anti-HA) staining of cells treated with TAT-DEDSH2, or TAT-DEDR86K, and confirms all cells take up the polypeptides.

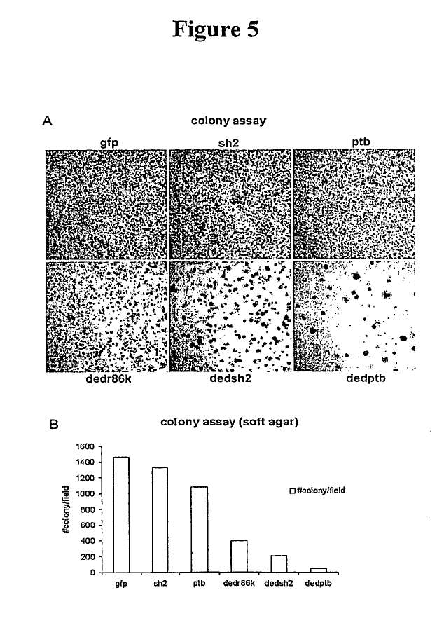

To access the ability of DEDSH2 and DEDPTB to block anchorage independent growth, Ntr2 cells were treated with the various adenoviruses (MOI 10:1) for 1 hour and then harvested. 10⁶ cells/plate were grown in soft agar for a period of 3 weeks. As shown in FIG. 5A, treatment of Ntr2 cells with either DEDSH2 or DEDPTB dramatically inhibited the anchorage independent growth of Ntr2 compared to controls. The colonies were microscopically counted to determine the number of colonies per field (FIG. 5B). It was estimated that there is at least a 100 fold decrease in colony forming ability of cells expressing DEDSH2 and a 1000 fold decrease in colony forming ability of cells expressing DEDPTB. However, due to the densitiy of colonies it was not possible to obtain accurate numbers for GFP, SH2, PTB, and DEDR86K treated cells, and the actual number of colonies is likely much greater.

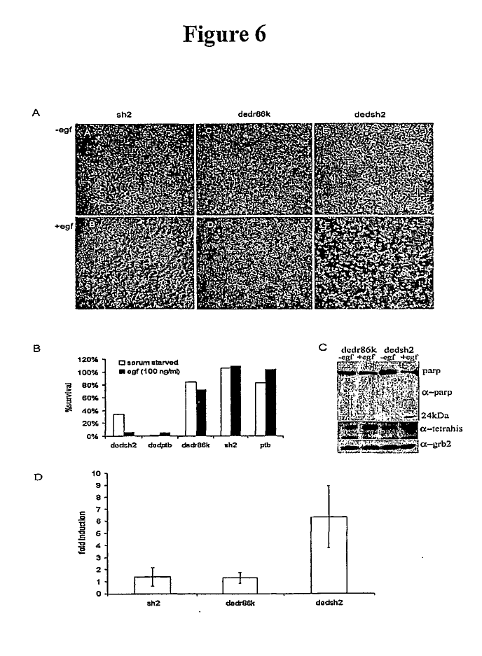

To further characterize the dependence of apoptosis on RTK signaling, a nasal pharyngeal carcinoma (npc) cell line (TWO3) was examined. TWO3 cells are derived from an Epstein-Barr virus (EBV) positive carcinoma, however, during ex vivo passages, they have lost EBV genomes, and are only weakly transforming. These cells express epidermal growth factor receptor (EGFR) and can be induced by EGF in the media. The cells were used to examine the dependence of apoptosis on RTK activitity. NPC cells were treated with SH2, DEDR86K, or DEDSH2 adenoviruses, and serum starved overnight. Cells were then stimulated with serum free media (EGF) or media containing different amounts of EGF (0-100 ng/ml). An increase in apoptosis was observed in DEDSH2 expressing cells that correlated with the amount of EGF in the media. This effect was most pronounced at high levels of EGF. In FIG. 6A, phase contrast micrographs of Npc cells, before and after EGF stimulation, are shown. An increase in apoptotic bodies was observed following EGF treatment in cells expressing DEDSH2. Apoptotic stimulation was not seen in cells expressing SH2 domain alone, or in cells expressing DEDR86K. The survival of Npc cells, following EGF stimulation, was quantified by crystal violet staining of surviving (adherent) cells 48 hours after treatment (24 hours after stimulation). As shown in FIG. 6B, EGFR stimulation leads to a decrease in survival of DEDSH2 expressing cells. DEDR86K expressing cells displayed a modest decrease in survival, whereas cells expressing SH2 or PTB domains alone did not show any decrease in survival. The effect of DEDPTB expression was also tested in this system. Unfortunately, DEDPTB expression was lethal to Npc cells and did not show any induction with EGF treatment.

Apoptosis is characterized by the activation of caspases that cleave substrate proteins at specific sites. The unique degradation pattern of caspase targets can be used to monitor activation of downstream effector caspases, and as a marker of cells undergoing apoptosis. To confirm that Npc cells expressing DEDSH2 were dying of apoptosis, lysates of Npc cells expressing DEDR86K, or DEDSH2 were analyzed by western blotting with anti-poly (ADP-ribose) polymerase (parp). Parp is a 116 kDa nuclear protein that is degraded by caspase 3 into 24 and 89 kDa fragments during apoptosis. As shown in FIG. 6C EGF stimulation (100 ng/ml, 4 hours) leads to parp cleavage in the presence of DEDSH2 but not DEDR86K(top panel). Blots were also probed with tetra-his antibody to confirm expression of DEDR86K, and DEDSH2 in these cells (middle panel). Reprobing of the membrane with anti-Grb2 confirmed equal loading of samples. Recruitment of DEDSH2 adaptors to activated EGFR is predicted to lead to activation of caspase 8. To examine the status of caspase 8, lysates from Npc cells expressing SH2, DEDR86K, or DEDSH2 were collected, and the caspase 8 activity was measured. As predicted from the model, stimulation of Npc cells with EGF leads to a 6 fold induction of caspase 8 activity in DEDSH2 expressing cells (FIG. 6D).

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the domains, cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

TABLE 1
Overexpression or mutation leading to constitutive activation is
implicated in many types of malignancies.

| | Involvement in cancer (activating mutation or |
|---|---|
| RTK | overexpression) |
| Tie-1 | Angiogenesis |
| Tek/Tie-2 | Angiogenesis/venous malformations |
| Flt-1; Flk; EGFR3 | Vasculogenesis/angiogenesis |
| EGFR/ErbB | Glioblastoma |
| ErbB2/neu | Breast and ovarian cancer; human adenocarcinoma |
| | of the salivary gland; promyelocytic leukemia; |
| | prostate cancer |
| PDGFR | Melanoma |
| ErbB3 | Breast cancer |
| Ret | Multiple endocrine neoplasia type IIA and IIb; |
| | Hirschsprung disease; medullary thyroid carcinoma; |
| | papillary carcinoma |
| Met/HGFR | Papillary renal carcinoma; hepatocellular carcinoma |
| Kit | Gastrointestinal stromal tumours; sporadic |
| | mastocytosis; acute myeloid leukemia |
| Alk | Large cell lymphoma or Alk lymphoma |
| Axl | Chronic myeloid leukemia |
| TrkA | Hematological/solid tumours, papillary thyroid |
| | carcinoma |
| FGFR1 | Stem cell leukemia/lymphoma syndrome; acute |
| | myelogenous leukemia |
| FGFR2 | Osteosarcoma in rats |
| FGFR3 | Multiple myeloma |
| Bcr-Abl | Chronic myelogenous leukemia |

We claim:

1. A method for modifying a RTK/PTK signal in a cell to an apoptotic signal in the cell comprising linking a RTK/PTK signaling pathway that transduces the RTK/PTK signal in the cell to an apoptotic signaling pathway that transduces the apoptotic signal in the cell, thereby modifying the RTK/PTK signal in the cell.

2. A method for activating an apoptotic signal in a cell comprising linking a RTK/PTK signaling pathway that transduces a RTK/PTK signal in the cell to an apoptotic signaling pathway that transduces an apoptotic signal in the cell thereby initiating the apoptotic signal in the cell.

3. A method as claimed in claim 1 or 2 wherein the RTK/PTK signaling pathway is linked to an apoptotic signaling pathway in the cell by creating a new signaling pathway in the cell comprising signaling molecules of an RTK/PTK signaling pathway that transduce a RTK/PTK signal, and signaling molecules of the apoptotic signaling pathway that promote apoptosis.

4. A method as claimed in claim 1 or 2 wherein a RTK/PTK signaling pathway in the cell is linked to an apoptotic signaling pathway in the cell using a domain of a signaling molecule that regulates the RTK/PTK signal and a domain of a signaling molecule that promotes apoptosis.

5. A method as claimed in claim 2, 3, or 4 wherein the RTK/PTK signaling pathway is a mitogenic signaling pathway, preferably an oncogenic signaling pathway, and the RTK/PTK signal is a mitogenic signal preferably an oncogenic signal.

6. A method as claimed in claim 1 or 2 wherein a RTK/PTK signaling pathway in the cell is linked to an apoptotic signaling pathway in the cell by coupling a domain of a signaling molecule that regulates the RTK/PTK signal and a domain of a signaling molecule that promotes apoptosis in the cell.

7. A method as claimed in claim 6 wherein the domains are coupled in situ.

8. A method as claimed in claim 1 or 2 comprising administering to the cell a domain of a signaling molecule that regulates an RTK/PTK signal and a domain of a signaling molecule that promotes apoptosis in the cell, in amounts effective to change the RTK/PTK signal to an apoptotic signal in the cell.

9. A method as claimed in claim 1 or 2 comprising administering to the cell a chimeric protein comprising a domain of a signaling molecule that regulates an RTK/PTK signal and a ligand-binding domain, and a chimeric protein comprising a domain of a signaling molecule that promotes apoptosis in the cell and a ligand-binding domain, in amounts effective to change the RTK/PTK signal to an apoptotic signal in the cell.

10. A method for enhancing apoptosis induced by a tumor necrosis factor (TNF)-family ligand comprising administering to a cell an amount of a domain of a signaling molecule that regulates a RTK/PTK signal in a cell and a domain of a signaling molecule that promotes apoptosis in the cell.

11. An isolated molecule comprising a domain of a signaling molecule that regulates a RTK/PTK signal in a cell and a domain of a signaling molecule that promotes apoptosis in the cell.

12. An isolated molecule as claimed in claim 11 wherein the domain that regulates a RTK/PTK signal is a domain that binds to a phosphotyrosine-containing protein, preferably an SH2 domain or a PTB domain, and the domain that regulates an apoptotic signal is a death or death effector domain, preferably a death effector domain of an adaptor protein.

13. An isolated molecule as claimed in claim 11 which is a Death Effector Domain-Src Homology 2 domain (DED-SH2).

14. An isolated molecule as claimed in claim 11 which is a Death Effector Domain-pTyr binding domain (DED-PTB).

15. An isolated molecule as claimed in claim 11 comprising a Grb2 SH2 domain or a PTB domain of Shc, and the death effector domain of FADD.

16. An isolated molecule as claimed in claim 11 in association with a caspase.

17. An antibody specific for a molecule as claimed in any preceding claim.

18. A nucleic acid molecule encoding an isolated molecule as claimed in any preceding claim.

19. An isolated nucleic acid molecule comprising a sequence encoding a domain of a signaling molecule that regulates a RTK/PTK signal in a cell and a sequence encoding a domain of a signaling molecule that promotes apoptosis in the cell.

20. A vector comprising a nucleic acid molecule as claimed in claim 18 or 19.

21. A host cell comprising a vector as claimed in claim 20.

22. A process for preparing a protein comprising: (a) transferring a vector as claimed in claim 19 into a host cell; (b) selecting transformed host cells from untransformed host cells; (c) culturing a selected transformed host cell under conditions which allow expression of the molecule and (d) isolating the protein.

23. A recombinant protein prepared using a method as claimed in claim 22.

24. A pharmaceutical composition, which contains pharmaceutically effective amounts of an isolated molecule as claimed in any of the preceding claims, or a nucleic acid molecule encoding an isolated molecule as claimed in any of the preceding claims, and a suitable pharmaceutical carrier or delivery system.

25. A method of treating disease conditions where affected cells have a defective RTK or PTK or a defective ligand for an RTK or PTK comprising linking a RTK/PTK signaling pathway in the cell to an apoptotic signaling pathway in the cell to activate the apoptotic signal in the cell.

26. A method as claimed in claim 25 wherein an effective amount of a domain of a signaling molecule that regulates a RTK/PTK signal in a cell and a domain of a signaling molecule that regulates an apoptotic signal to promote apoptosis in the cell are administered.

27. A method as claimed in claim 25 or 26 wherein the RTK or PTK is a mutated RTK or PTK or over expressed RTK or PTK.

28. A method as claimed in claim 25 or 26 wherein a ligand for an RTK is mutated or over expressed.

29. Use of a molecule or nucleic acid molecule claimed in any preceding claim in the preparation of a medicament for treating or preventing disease conditions where affected cells have a defective RTK or PTK or a defective ligand for the RTK or PTK.


SEQUENCE LISTING (10 sequences)

SEQ ID NO: 1 - DNA, 537 bp, Artificial Sequence
  DEDSH2 adaptor molecule
atggacccattcctggtgctgctgcactcgctgtccggcagcctgtcgggcaacgatctgatggagctcaagttcttgtgccgcgagcgcgtgagcaaacgaaagctggagcgcgtgcagagtggcctggacctgttcacggtgctgctggagcagaacgacctggagcgcgggcacaccgggctgctgcgcgagttgcttgcctcgctgcgccgacacgatctactgcagcgcctggacgacttcgagaattcgtggttttttggcaaaatccccagagccaaggcagaagaaatgcttagcaaacagcggcacgatggggcctttcttatccgagagagtgagagcgctcctggggacttctccctctctgtcaagtttggaaacgatgtgcagcacttcaaggtgcttcgagatggagccgggaagtacttcctctgggtggtgaagttcaattctttgaatgagctggtggattatcacagatctacatctgcttccagaaaccagcagatattcctgcgggacatagaacag

SEQ ID NO: 2 - protein, 179 aa, Artificial Sequence
  DEDSH2 adaptor molecule
MDPFLVLLHSLSGSLSGNDLMELKFLCRERVSKRKLERVQSGLDLFTVLLEQNDLERGHTGLLRELLASLRRHDLLQRLDDFENSWFFGKIPRAKAEEMLSKQRHDGAFLIRESESAPGDFSLSVKFGNDVQHFKVLRDGAGKYFLWVVKFNSLNELVDYHRSTSASRNQQIFLRDIEQ

SEQ ID NO: 3 - DNA, 894 bp, Artificial Sequence
  DEDPTB adaptor molecule
atggacccattcctggtgctgctgcactcgctgtccggcagcctgtcgggcaacgatctgatggagctcaagttcttgtgccgcgagcgcgtgagcaaacgaaagctggagcgcgtgcagagtggcctggacctgttcacggtgctgctggagcagaacgacctggagcgcgggcacaccgggctgctgcgcgagttgcttgcctcgctgcgccgacacgatctactgcagcgcctggacgacttcgactcgagaatgaacaagctgagtggaggcggcgggcgcaggactcgggtagaagggggccagctggggggcgaggagtggaccagacacgggagctttgtcaataagcccacacgaggctggctgcatcccaacgacaaagtcatgggacctggggtttcctacttggttcggtacatgggctgtgtggaggtcttacagtcaatgcgagcccttgacttcaatacccggactcaggtcaccagggaggccatcagtttggtgtgtgaagctgtgcctggtgccaaaggggcgacaaggaggagaaagccttgtagccgcccactcagctccatcctggggaggagtaacctgaagtttgctggaatgccaatcactctcactgtgtctaccagcagccttaacctcatggcagccgactgcaaacagatcattgccaaccatcacatgcaatctatctctttcgcgtccggtggggatccggacacagctgagtatgttgcctatgttgccaaagaccctgtgaatcagagagcctgccatatcctggagtgtcctgaagggcttgctcaggatgtcatcagcaccatcgggcaggcctttgagttgcgcttcaaacagtatctcaggaatccaccgaagctggtc

SEQ ID NO: 4 - protein, 298 aa, Artificial Sequence
  DEDPTB adaptor molecule
MDPFLVLLHSLSGSLSGNDLMELKFLCRERVSKRKLERVQSGLDLFTVLLEQNDLERGHTGLLRELLASLRRHDLLQRLDDFDSRMNKLSGGGGRRTRVEGGQLGGEEWTRHGSFVNKPTRGWLHPNDKVMGPGVSYLVRYMGCVEVLQSMRALDFNTRTQVTREAISLVCEAVPGAKGATRRRKPCSRPLSSILGRSNLKFAGMPITLTVSTSSLNLMAADCKQIIANHHMQSISFASGGDPDTAEYVAYVAKDPVNQRACHILECPEGLAQDVISTIGQAFELRFKQYLRNPPKLV

SEQ ID NO: 5 - DNA, 18 bp, Artificial Sequence
  primer dedf1
cgagatttacccatggac

SEQ ID NO: 6 - DNA, 18 bp, Artificial Sequence
  primer dedr1
gtcaaatgccacctgcag

SEQ ID NO: 7 - DNA, 30 bp, Artificial Sequence
  primer dedf2
ttaagcttatggacccattcctggtgctgc

SEQ ID NO: 8 - DNA, 30 bp, Artificial Sequence
  primer dedr2
agaattctcgaagtcgtccaggcgctgcag

SEQ ID NO: 9 - DNA, 28 bp, Artificial Sequence
  primer SH2f1
aagaattcgtggttttttggcaaaatcc

SEQ ID NO: 10 - DNA, 27 bp, Artificial Sequence
  primer SH2r1
aatctagactgttgtatgtcccgtaag